(19) United States
(12) Patent Application Publication
(10) Pub. No.: US 2004/0054380 A1
(43) Pub. Date: Mar. 18, 2004

(54) Enhancing human ventricular contractility, perfusion, and rhythm by enhancing laminar coordination of ventricular myocardial repolarization by the His-Purkinje system

(76) Inventors: David Franklin Craig, Burnsville, NC (US); William David McGuinn JR., Columbia, MD (US)

Correspondence Address: David Franklin Craig, 838 J.R. Pate Road, Burnsville, NC, 28714, US

(21) Appl. No.: 10/241,857

(22) Filed: Sep. 13, 2002

(51) Int. Cl. A61N 1/36

(52) U.S. Cl. 607/9

(57) ABSTRACT

New in the art of medicine in this invention is the ability to improve cardiac output and transmural ventricular perfusion and diminish ventricular arrhythmias in the human heart by correcting a common defect in electrical regulation of the systole of the ventricles, this being a defect in a previously unknown mechanism, namely laminar coordination of ventricular repolariation by the His-Purkinje system.

### CROSS-REFERENCE TO RELATED APPLICATIONS

Not applicable.

### STATEMENT REGARDING FEDERALLY SPONSORED RESEARCH OR DEVELOPMENT

There was no federal funding or sponsorship of this research and development.

### REFERENCE TO A MICROFICHE APPENDIX

Not applicable.

### BACKGROUND OF THE INVENTION

This invention pertains to the practice of human medicine, and particularly to the management of diseases of the human heart. It pertains to using the principles of cardiac electrophysiology for the treatment of clinically demonstrable heart disease.

Reference is hereby made to the article “A mechanism for laminar coordination of ventricular myocardial repolarization and contraction by the His-Purkinje system” by David Franklin Craig MD and William David McGuinn Jr. PhD, which is included with this application. Reference is also made to the past articles cited in the reference section of the above article, which detail the prior history of cardiac electrophysiology and the absence of any previous awareness of the concept of laminar coordination.

Previous mistaken attribution of regulation of ventricular repolarization to thermal gradients or ischernia has resulted in an inability to mitigate clinically the problems which result from defective regulation of ventricular repolarzation, including heart failure, subendocardial ischemia, and ventricular arrhythmias. This invention now provides the means to mitigate these.

### BRIEF SUMMARY OF THE INVENTION

The nature of this invention is the enhancement of the process of laminar coordination of ventricular myocardial repolarization. This is done by stimulating or enhancing the processes in the Purkinje cells which effect sustained depolarization of the myocardial cells, or enhancing the responsiveness of the myocardial cells to this regulation.

The object of this invention is to enhance contractility, suppress ventricular arrhythmias, and enhance transmural perfusion in the ventricles of the human heart.

There exist and have existed problems in clinical medicine with deficiencies of contractility and perfusion, and occurrence of arrhythmias, in the ventricles of the human heart which persist even in the absence of vascular obstruction or after correction of obstruction. This invention provides the ability to compensate in part for the sequelae of vascular obstruction in the coronary arteries, or to improve function when vascular lesions are not present,

### BRIEF DESCRIPTION OF THE SEVERAL VIEWS OF THE DRAWING

Not applicable.

### DETAILED DESCRIPTION OF THE INVENTION

The nature of this invention is the enhancement of the process of laminar coordination of ventricular myocardial repolarization, as described in the article “A mechanism for laminar coordination of ventricular myocardial repolarization and contraction by the His-Purkinje system” by David Franklin Craig MD and William David McGuinn Jr. PhD, which is included with this application.

This enhancement may be achieved by any means, when used for this purpose. The primary and best means will be by applying electronic pacemakers and leads to the portions of either or both human ventricles which are to have their regulating portion of the His-Purkinje system enhanced, or by using drugs which regulate or enhance the cellular or subcellular structures and processes in Purkinje cells or ventricular myocardial cells by which laminar coordination is produced.

There has been no previous awareness of the role of the His-Purkinje system in regulating ventricular repolarization, as detailed in the afore-mentioned article. The His-Purkinje system has previously been thought by all authorities to only initiate ventricular depolarization, and has not been thought to regulate the ongoing state of depolarization or to regulate the initiation of repolarization. The concept and process claimed as this invention, of enhancing the process of laminar coordination, can serve to increase contractility in the human heart, as well as improving transmural perfusion in the ventricles and reducing the incidence of cardiac arrhythmias of ventricular origin. This process can improve these factors without imposing additional stresses on the heart, which has not previously been possible.

Introduction:

In the 1930's, Frank Wilson observed that the T wave of the normal electrocardiogram (EKG) should point in a direction opposite the R wave (1). This is because the QRS complex represents the wave of electrical depolarization and the T wave that of repolarization through the myocardium. Thus, the voltages should be electrically opposite if the path of depolarization follows that of repolarization. Yet, the T wave normally points in the same direction as the R wave. Wilson attributed the upright T wave to “local factors”, but was never able to define these.

Current textbooks list the default explanation for upright T waves as being due to transmural ischemia, indicating that the issue still is felt to have some importance. The last attempt to change this doctrine was by Prinzmetal and others in 1957, invoking temperature differentials. This apparently was unable to displace what remains the default explanation, offered by Grant and Estes in a 1951 text, of ischemia. They recognized the need for a mechanistic explanation of the genesis of the physiologic upright T wave. Frank Wilson had visited the temperature differential theory in a 1923 paper, but correctly realized the limitations of the concept, and formulated the question quite correctly in a 1931 paper as being what local effect was involved, admitting explicitly that the source of the effect was unknown. Wilson's attempts to explain repolarization changes in terms of a ventricular gradient, and dividing T changes into primary and secondary nature, appear to be an unsuccessful effort to link T wave changes to passive conduction properties of myocardium. The inability to define the mechanism of upright T waves has frustrated attempts to formulate a rigorous comprehensive theory of human electrocardiography as Wilson pursued in the 1920's and 30's. After 1957, theory was abandoned and EKG's came to be analyzed on a solely empirical basis derived from anatomic pathology correlations. This surrendered the opportunity to predict pathologies that cannot yet be measured. Here, we revisit the nature of the local factors at work in the layers of the human ventricle, in the hope of regaining such predictive ability.

Methods:

Electrocardiograms obtained in the course of clinical practice were reviewed for patterns of transition over time or within the same tracing in intraventricular conduction, and relationship of QRS complex morphology to changes in T wave morphology. The literature was reviewed for mechanisms of the genesis of the normal and abnormal electrocardiogram in regard to conduction distal to the atrioventricular node, patterns and distribution of ventricular repolarization activity, and patterns of disease and symptomatology related to abnormal findings. Previously proposed mechanisms were systematically eliminated based on known cardiac physiology.

Findings:

In an isolated strip of myocardium the wave of movement of repolarization of myocardial cell transmembranous electrical potential follows the path of depolarization, i.e., the first cells depolarized are the first cells repolarized. This deflects the externally measured electrical field opposite to the field measured during depolarization. This is also seen in a skin surface electrode in the electrocardiogram, in the presence of left ventricular hypertrophy (LVH), or in left bundle branch block (LBBB). In these abnormalities, the T wave deflects downward, opposite the R wave.

This comparison to direct transmural leads is best illustrated in consistently R dominant leads such as I and V6. These closely resemble the waveforms expected from a transmural lead on the ventricular free wall, with the normal septal Q wave serving as the main reminder that the comparison is not complete, and that other muscle areas do contribute to the complexes. This allows the use of the noninvasive standard EKG to screen large numbers of present and historical subjects for clues to pathophysiology.

In a normal supraventricular beat with normal bundle conduction, the T wave deflects in the same direction as the R wave. In this physiologically normal state, repolarization of the ventricular myocardium occurs from the subepicardial muscle toward the subendocardial muscle, opposite to that seen in isolated muscle preparations or in LVH. That is, the first cells depolarized are the last cells repolarized.

The mechanism of this reversal in the direction of the repolarization wave has been supposed to be relative stress and ischemia in the subendocardial muscle cells due to intraventricular pressure and to the increased distance from the epicardial vasculature (2,3). This appears unlikely, since left intraventricular pressure falls well below aortic diastolic pressure immediately after aortic valve closure, and should have little effect on coronary artery flow during all of diastole, the only period when coronary artery perfusion occurs. Additionally, the concept that a little ischemia causes upright T waves but a more severe ischemia causes inverted T waves suggests that two mechanisms are involved, not one. We delineate here a different mechanism.

We occasionally see in clinical practice that an intermittent LBBB can occur, even to the point of alternating blocked and normally conducted supraventricular beats, with the T waves being in the same direction as the R waves after the normal beats, and opposite the R waves after the LBBB beats (4). This is an experiment of nature with important lessons. This rapid alternation in the direction of repolarization suggests that ischemic or endocrine control of the direction of repolarization cannot be the controlling factor, nor regional variations in temperature (5,6). Only an electrical mechanism of control can alternate so rapidly.

We propose that a voltage-clamp-like effect occurring in the subendocardial region of the ventricular muscle locks the ventricular myocardium in depolarization. The muscle region farthest from the voltage clamp (the subepicardial region) breaks through to repolarization first, with the repolarization wave spreading toward the subendocardial region. This repolarization wave finally overcomes the voltage clamp, or blends in with its cessation.

This illustration of alternating LBBB and normal beats serves to reveal the location and source of the voltage clamp effect. When the bundle acts, we see upright T waves. When the bundle is inactive, as seen in the QRS by prolongation, we see downward T waves. Only the action of the His-Purkinje system is altered between the alternating normal and LBBB beats, all other anatomic and chemical factors and temperature are held the same. This shows the Purkinje end fibers penetrating the subendocardium as the source of the voltage clamp. The Purkinje network not only initiates depolarization, as is recognized, but enforces sustained depolarization in a coordinated manner, and is Wilson's unfound “local factor”. This finding is better illustrated by LVH, wherein the inverted T waves follow QRS complexes traveling directly outward through the left ventricular wall, but no instance of intermittent LVH conduction delay has been observed which could display the changes more vividly.

The inability to see Purkinje function in isolated strips of myocardium is probably due to a combination of using small segments of myocardium which do not include sufficient Purkinje cells to provide electrical activity, and setting stimulation levels at minimal voltages that excite myocardial cells but not Purkinje cells.

The inductive reasoning involved above leads by deduction to several corollary concepts which were tested against published experimental findings, with good agreement.

The Purkinje end fibers penetrate about one third of the way into the normal 11 mm thickness of the normal ventricular wall (7,8). In this 3 to 4 mm deep region the voltage clamp effect is strongly implemented for the duration of the roughly 0.40 second action potential of the Purkinje fibers. This coincides with the roughly 0.4 second Q-T interval (9), thus demonstrating the tracking of myocyte action potential duration with the action potential duration of the adjacent purkinje fibers rather than their own native unregulated duration as measured in the laboratory. Thus the action potential of the subendocardium is locked in place, probably by the enforced extracellular negative charge sustained by the Purkinje action potential, until the relatively unclamped subepicardial, and then the relatively less-clamped midmyocardial, regions have successively repolarized. This results in a reversal of the direction of repolarization from the direction of depolarization. The action or inaction of this mechanism allows the rapid alternation of the direction of repolarization, and the resulting direction of the T wave deflection, even from beat to beat.

The Purkinje action potential would be wasteful of energy if it serves only to activate the myocardium, and would better resemble the brief spike of a neuron. The subendocardial myocyte action potential tracks that of the Purkinje cells, reverting to its native length if there is no Purkinje activation. These findings are causal, not coincidental. See Table I.

Mechanism:

The prolongation of the depolarization of the Purkinje-clamped myocytes is essentially a prolongation of the plateau phase of the action potential. Mid and late plateau is largely sustained by the action of the sodium-calcium exchange pump.(10) This electrogenic pump functions during plateau by exchanging an inward flow of sodium ions, downhill on the sodium concentration gradient, with an outward flow of calcium ions, with a resultant net inward current and an elevated transmembrane voltage, sustaining depolarization. This current is energetically sustained and augmented by low extracellular calcium concentration, which makes outward flow of calcium more energetically favorable, and net inward current flow thereby more favorable. This shows that the Purkinje fibers can exert the voltage clamp by maintaining low calcium concentration in the extracellular fluid (ECF). Reduction in the volume of ECF during contraction should reduce the quantity of calcium that has to be taken up by the Purkinje cells to effect voltage clamping. Noble has demonstrated a “restricted extracellular space” for potassium in the ventricular extracellular fluid(11); this same restricted physical space aids in the maintenance of hypocalcemia adjacent to the myocytes by the Purkinje cells. Clinically, we already know that hypocalcemia prolongs the QT interval (12), which is also to say that it prolongs the action potential and the depolarization of myocytes. Purkinje mediated calcium depletion may also exert some of its effect on the myocytes by delaying or reducing the repolarizing currents, specifically the delayed rectifier potassium current (13), since Yokoshiki et al (14) report a lesser expression of transient outward current channels in subendocardium than in subepicardium. It should be possible by conventional electrophysiological methods or by vector mediated receptor expression techniques to determine if and how ECF calcium depletion can alter the delayed rectifier. We might expect the delayed rectifier channels and sodium-calcium exchange pumps in Purkinje cells to show structural differences or altered expression from the ones in ventricular myocytes, which would result in their different activities.

Consequences of Mechanism and Known Laboratory Confirmation of Consequences:

The subepicardial muscle typically provides the end of depolarization voltage about 0.06 second after the subendocardial muscle starts the R wave. Then the subepicardium starts the T wave about 0.25 second later, as its action potential (as reported by Sperelakis) (15) ends, or 0.31 second after the start of the R wave on the EKG. The subendocardial muscle then provides the end of the repolarization voltage, and the end of the T wave, about 0.1 second after the start of the T wave. This is after the 0.40 second action potential of the Purkinje fibers (per Sperelakis) has ended, the voltage clamp with it. Thus, we see proven in a normal beat on EKG the synchronization of the subendocardium with the Purkinje action potential.

A depolarization originating in the myocardium tends not to excite the Purkinje cells in a retrograde manner, suggesting a degree of autonomy in the Purkinje cells consistent with a voltage clamping regulatory function. We see this clinically in a premature ventricular contraction (PVC), which would have QRS duration similar to a Purkinje-fired beat if conduction spread from the original focus through the rapid pathway of the Purkinje fibers, but instead is actually quite prolonged. Hoffmann et al (16) have provided evidence of this relative resistance to retrograde excitation at the cellular level. This suggests that excitation of the His-Purkinje system, at some point distal to the atrioventricular (A-V) node, becomes preferentially dependent on activation directly from adjacent Purkinje cells, probably through the gap junction attachments. Direct conduction could account for the relatively greater speed of conduction in the His-Purkinje system as reported by Sperelakis, as ionic flows leap from gap junction to gap junction in a manner very analogous to the leap between nodes of Ranvier in myelinated nerve fibers The absence of transverse tubules in the Purkinje fibers (17) may contribute to resistance to retrograde excitation. Resistance to retrograde conduction is known to occur in an analogous way at the A-V node according to Akhtar et al (18), where it plays a role in interrupting the path of potential reentrant arrhythmias. Thus, we may think of the intersection of Purkinje end fibers with ventricular myocardium as a dispersed signal junction working to isolate ectopic foci in the myocardium. A fusion beat, where a PVC blends into a conducted beat, is another experiment of nature where the resistance to retrograde conduction leaves the Purkinje fibers polarized and available to spread the supranodal signal even after the ventricular focus has fired. This tends to contain the ectopic beat and reset the myocardium.

The successful use of overdrive pacing in suppressing ventricular arrhythmias suggests that Purkinje refractoriness plays a role in the genesis of such rhythms. As a scroll wave circuits the ventricles with time periods as short as 0.25 second, the Purkinje system appears to remain refractory to retrograde excitation over the short period of adjacent myocardial depolarization. This probably requires suppression of the SA or AV node by vagal reflexes. This then permits the scroll wave to act in a uniform conductive medium of myocardium, while the Purkinje network sits idle. When overdrive pacing is used to excite the Purkinje system from the endocardial surface, the system imposes a prolonged refractory period on the adjacent myocardium throughout the paced ventricle, which traps and suppresses the autonomous ventricular rhythm as the scroll wave meets tissue which is still refractory. The scroll wave then simply extinguishes. Activation of the Purkinje system at a sufficient rate, spreading within and throughout the system rapidly, is a part of the physiologic suppression of such abnormal rhythms, but has not been appreciated empirically. Employing this understanding may offer a tremendous opportunity to improve the clinical suppression of ventricular arrhythmias. Activating both bundle branches by overdrive pacing can potentially halt established ventricular dysrhythmias in any patient in which temporary or permanent leads are present. Implanted pacemaker design could be altered to allow for sensing such rhythms and automatically start a sequence of overdriving then slowing to normal rates. This would be much less stressful to the patient than external or internal defibrillation or cardioversion. Access to the Purkinje system from endocardial leads would reduce the energy levels involved and avoid some of the potential for cellular injury. This avoidance of injury could also be very helpful in reducing the risk from thrombolytics when dysrhythmia occurs in the setting of acute myocardial infarction.

El Sherif et al (19) noted that most initial foci for ventricular tachyarrhythmiias originate in the subendocardium, and Yokoshiki et al report that these arrhythmias are more common in the presence of LVH. The loss of voltage clamping would have precisely this effect. In control theory in engineering disciplines, the broadest range of activity of a regulated mechanism is obtained by operating in the middle of the linear range of the output variable. Responsiveness to control is greatly enhanced by conditions that yield great instability of the output variable if the regulating input should be absent. Thus if, teleologically speaking, the regulation of repolarization of ventricular myocardium should be optimized, the loss of that active regulation under control theory would predict an unstable and probably chaotic pattern of repolarization in the previously regulated subendocardium. Zabel et al (20) have implicated disparate refractory periods in localized areas of subendocardium in the origin of ventricular tachyarrhythmias, and these are precisely what is predicted from loss of coordination of repolarization in the ventricle by the Purkinje network, under the voltage clamp concept.

Magnesium sulfate is one of the more useful drugs for treating ventricular arrhythmias (21). Like calcium, magnesium is a divalent cation. This cation can alter this system that is primarily regulated in the normal state by the calcium cation; increasing magnesium prolongs the refractory period of ventricular myocytes (22). The antiarrhythmic effect of magnesium ion may occur because the magnesium ion concentration is forcing the myocyte channels toward the more saturated and uniform response of their upper (i.e. more prolonged) activity range, and out of the more unstable midrange, when calcium depletion by the Purkinje cells has been lost. This restoration of uniformity, by saturation, reduces arrhythmogenesis even though prolonged depolarization and QT interval is ordinarily thought of as being intrinsically arrhythmogenic. Defining the effect of extracellular magnesium concentration on channel and pump activity should accompany investigations of the role of calcium in the Purkinje voltage clamp. This understanding of the voltage clamp mechanism in preventing arrhythmias may lead to the development of technologies that decrease susceptibility to ventricular arrhythmias.

Charpentier et al have noted that afterdepolarizations in ventricular myocardium are increased in the presence of high levels of catecholamines (23); normal contractility due to normal voltage clamping, as discussed below, may help prevent congestive heart failure (CHF) from triggering release of compensating catecholamines and the arrhythmogenic afterdepolarizations.

Hamlin et al (24) note that in the pig the Purkinje fibers reach nearly to the epicardium. The voltage clamp concept predicts that the Purkinje system of the pig heart would better suppress ectopic foci in the pig ventricle. But, this would also reduce the utility of the pig as a model for human arrhythmias and antiarrhythmics. Carnivores and primates may be accurate models, and not ungulates.

Even the cell structure of Purkinje cells is consistent with the function of a voltage clamp. The large size of Purkinje cells gives a greater volume in which to disperse the prolonged influx of calcium ions, and the gap junctions offer the means to disperse any regional increases in calcium uptake farther across the Purkinje network. The absence of large numbers of contractile elements also prevents those elements from being damaged by the prolonged high levels of calcium in the intracellular space. It is not presently apparent whether the sarcoplasmic reticulum (SR) functions differently in Purkinje cells compared to ventricular myocytes, but teleologically it would be advantageous if the SR either did not add to intracellular fluid calcium ion concentration or even helped to sequester it during systole in the case of Purkinje cells. The absence of T tubules may serve to uncouple the SR from the action potential at the sarcolemma, and allow the SR to provide such sequestration.

The sustained voltage clamp effect of the Purkinje network is also teleologically attractive in its effect on contractility. From the time the subepicardial muscle depolarizes, at 0.06 second after the start of the R wave, 100 per cent of the thickness of the ventricular muscle is recruited for contraction for 0.25 second. See FIG. 1. This occurs since contraction essentially coincides with depolarization in a given myocyte, and with the QT interval on EKG. This allows a sustained full-force contraction to aid in the pumping of blood outward from the ventricles. It also allows time for maximal pump work to be extracted from the energy expended on myofibril contraction, with increased energy efficiency. The fact that the interventricular septum is excited by Purkinje end fibers in the left ventricular subendocardium indicates that pump function in the more highly loaded left ventricle is favored by having this coordinated repolarization and contraction imposed on the septum from the left.

Consider the effect on contractility if the voltage clamp effect is lost. The subendocardium begins to repolarize, and thereby end contraction, before the subepicardium, thereby leaving a partial thickness of muscle to complete the work of pumping blood out to the aorta. We also see that the subepicardial repolarization, occurring at its usual time, now represents the end, not the beginning, of the T wave. This increases the metabolic stress on the subepicardium, and causes a more rapid falloff in intraventricular pressure due to a falloff in recruitment of contractile elements, with less total stroke volume expressed. LVH especially produces this condition, where one sees a wall thickness of, for example, 22 mm instead of the normal 11 mm. This wall thickening creates a greater linear distance from the Purkinje end fibers to much of the myocardium, leaving more of it unclamped. The time to spread depolarization from subendocardium to subepicardium will roughly double, from the expected 0.06 second to 0.12 second. The action potential duration of unaltered myocardium is about 0.25 second. The unclamped subendocardium thus repolarizes as little as 0.13 second after depolarization of the subepicardium, giving only 0.13 second of full-force full-thickness contraction to effect stroke work, roughly half the normal duration. At the same time, the subepicardium must overcome the additional burden of the elastic recoil of the subendocardium, further reducing effective stroke work. This results in decreased energy efficiency compared to a normal beat in a nonhypertrophic ventricle. The total energy expended in the various layers of the thickened ventricular wall should be no less, and quite probably more, than in an unthickened properly coordinated wall. This greatly increases the risk of cellular injury. This effect is difficult to observe in vivo, since homeostatic mechanisms such as heart rate, preload, and catecholamine release intervene to normalize cardiac output. There is nonetheless a loss of reserve and increased stress on the myocardium.

Uncoordinated contraction is already understood to be produced in LBBB and in PVC's. In this case, the difference in time of contraction occurs laterally across the ventricular wall. Upshaw (25) notes this to reduce ejection fraction and therefore stroke volume by 13 percent in healthy hearts and 39 percent in the presence of ischemic disease. The loss of voltage clamping causes loss of coordination between different layers through the ventricular wall, by contrast. Thus, we can refer to this as laminar coordination or uncoordination, of both repolarization and contraction.

The surface EKG in far-advanced LVH gives evidence of additional trouble, with a broad QRS approaching 0.12 second duration slurring rapidly into an inverted T wave. This early T wave is a sign of an action potential duration in the subendocardium of only about 0.15 second instead of the normal 0.25 second, with even earlier loss of recruitment of subendocardial contraction, potentially even before subepicardial contraction begins, with reduction of even the peak of recruitment of contractile elements below 100 per cent. This would cause a still lower peak intraventricular pressure and an earlier falloff in pressure, reducing the area under the intraventricular pressure-time curve still further and reducing both stroke volume and energy efficiency.

We can occasionally see advancing LVH blend electrocardiographically into LBBB. We can explain this as increasing the distance of the non-voltage-clamped proportion of the ventricular wall muscle as the ventricle thickens during LVH. This will progressively prolong the QRS and invert the T wave. It may be that injury to the Purkinje system eventually completing the LBBB pattern on the EKG is actually caused by the stresses imposed on the ventricle, rather than being a primary event. Noma and Tsuboi (26) report gap junction resistance in Purkinje fibers to increase during acidosis; this could be one mechanism for Purkinje functional deterioration in LVH. Any stress leading to permanent reduction in the number of patent gap junctions could block or weaken Purkinje function despite retaining normal histology.

Part of the events in Purkinje-coordinated depolarization and repolarization is a set of time-varying cyclic pressure gradients in the layers of myocardium. Pressure gradients are associated with flow in liquids, moving from high pressure to low pressure at any instant. Therefore the blood or ECF in the ventricular wall must be obeying these pressure gradients and moving in a cyclic fashion. This may have some importance for transport of blood and oxygen through the wall to the subendocardium and Purkinje system. Long term changes in transmural perfusion secondary to loss of voltage clamping could cause significant damage to the Purkinje system.

The contractions of the layers of the ventricular wall must exceed the intraventricular pressure in order for blood to be pumped and constriction of the ventricular lumen to occur. In addition, the fibroelastic matrix of the ventricular wall also adds some degree of drag in order to be able to reexpand the ventricular lumen during diastole; this drag also raises the pressure within the wall itself above the pressure in the ventricular lumen. The pressure in the lumen of the coronary arteries must be lower than in the ventricle, or blood could not flow into the coronaries. Therefore during systole, up until the closure of the aortic valve, the left ventricular myocardium is necessarily unable to receive blood from the coronary arteries since it is at a higher pressure.

In the normal state with Purkinje activation and laminar coordination of contraction, the subepicardium relaxes, and should receive arterial inflow, before the subendocardium begins to relax. Thus, when the subendocardium begins to relax and allow inflow, the intramural arteries (27) should already be widely patent.

Contrast this with laminar uncoordination of contraction. As the subendocardium relaxes, it meets the intramural arteries occluded still by the pressure in the still-contracting subepicardium, much as the external pressure of a blood pressure cuff occludes the brachial artery. For a brief time, the only inflow to the subendocardium is venous effluent from the subepicardium. This effluent may fill a sufficient portion of the vascular and extracellular space such as to impair fresh arterial inflow after the subepicardium does relax. The ECF will be flushed with fresher material later in diastole, but to any degree that fresh inflow is delayed or impaired, metabolic impairment will be selectively imposed on the midmyocardium and especially the subendocardium and Purkinje system. This adds a mechanism of injury to the subendocardium and Purkinje system, completely independent of focal or generalized arterial occlusion. Upshaw notes a problem with false positive Thallium studies with septal ischemia despite normal coronary arteriography in LBBB. This anomaly suggests the transmural perfusion defect as described above. This defect can cause the appearance of small vessel disease despite actual normal anatomy.

This selective injury to the Purkinje system will deteriorate a poorly voltage-clamped system that shows only inverted T waves on EKG into a progressing LVH electrical pattern, then a completing LBBB as Purkinje function is totally lost, with further loss of stroke volume and work. It might be better to think of the repolarization and conduction changes seen with T flattening, T inversion, or LVH conduction delay on EKG as three stages of left bundle branch impairment or LBBI, and consider physical LVH indicated by traditional voltage criteria to be a different disease process. It should be pointed out here that all of the discussions of function and injury should apply to the right bundle branch and ventricle also, but the less loaded right ventricle less often presents dramatic findings clinically and is therefore somewhat neglected in this article.

The transmural perfusion defect during laminar uncoordination can explain the phenomenon of cardiac memory, wherein T wave inversion is sustained after a period of right ventricular pacing has ended. Subendocardial ischemia during pacing would cause changes in Purkinje cells, such as closure of gap junctions and decreased expression of calcium channel proteins in the sarcolemma and calcium pumps in the sarcoplasmic reticulum, that would only slowly return to normal states after normal Purkinje activation had resumed with cessation of right ventricular pacing. The continuation of T inversion should cause a positive feedback through Purkinje ischemia that would tend to weaken voltage clamping and further sustain the T inversion. The duration of the observable cardiac memory effect should have a lower limit largely determined by the rate of turnover and synthesis of the functional proteins involved. The development of the memory effect should similarly be closely related to turnover of those proteins, in terms of the duration of altered activation by pacing.

Teleologically, one might ask what functional advantage a human, or apparently carnivore, heart can have over that of a pig, by having only partial penetration of the ventricular wall by the Purkinje fibers as noted earlier. One answer is the effect on transmural perfusion as noted above. In the human, venous and ECF outflow from the subendocardium is enhanced at the onset of systole by the fact that the outer layers are not yet obstructing the venous channels by contracting. This reduces the volume of fluid trapped in the deep layers during systole, and reduces the amount of elastic remodeling that must occur during contraction and thickening of the ventricular wall. This reduces energy expenditure. Then, when diastole begins, the deep layers have a greater capacity to admit fresh oxygenated blood by virtue of having been squeezed relatively dry. The combination of greater mechanical energy efficiency and improved transmural perfusion should serve to increase physical endurance during flight or pursuit, and can well be considered to be evolutionarily advantageous.

Conducting system injury complicates primary vascular injury. The posterior division of the left bundle is supplied by branches from the right coronary and left anterior descending (LAD) arteries, and the anterior division is supplied by branches of the LAD alone (28). A relatively small infarct in the distribution of these arteries might not injure enough myocardium to be expected to badly impair left ventricular output. Yet this infarct may include either or both divisions of the left bundle, adding laminar or lateral uncoordination of contraction in part or all of the ventricular wall, which will then lead to progressing deterioration of pump work, CHF, and arrhythmias, even without further infarct. Therefore we might have predicted that anterior myocardial infarction (MI) from an LAD lesion, or inferior MI from a right coronary lesion with LBBB, would have elevated mortality rates compared to infarcts in other areas or without LBBB, as is empirically already known (29).

Discussion:

The concept of laminar coordination presented here is a hypothesis which requires laboratory confirmation to become strongly tenable and replace the current orthodoxy. Much of the underpinnings of this new theory have already been confirmed by the referenced laboratory and clinical findings, but investigational techniques must be altered to directly observe and confirm the theory. It is appropriate here to suggest the laboratory and clinical opportunities which become possible.

The loss of laminar pump coordination will be hard to measure experimentally, requiring separate time-graphed pressure transducers for various points within the ventricular wall. A wire with pressure transducers arrayed along it, pierced through the ventricular wall, may show the variations in time of the contraction at different depths. Voltage clamping itself may not be directly measurable, but only inferred from its effects on contraction. It may become possible, by selectively poisoning the Purkinje cells in laboratory animals, to progressively weaken the ECF calcium depletion and show the loss or restoration of voltage clamping more directly.

Trapping of ventricular ectopy by resistance to retrograde activation of the Purkinje system may be a measurable phenomenon in vitro. This also implies an ability to continue scavenging calcium from the local extracellular fluid until action potential terminates autonomously.

Protection of the Purkinje system through prevention of development of LVH represents a way to prevent occurrence of LBBB & CHF. An isolated area of simple T flattening or inversion on the EKG represents the earliest sign of local Purkinje dysfunction. This represents the loss of voltage clamping and laminar coordination locally, without loss yet of the already recognized depolarizing conduction. Even at this early stage, injury to Purkinje and other subendocardial cells may be occurring locally, due to impaired transmural perfusion due to laminar uncoordination. Valve replacement may need to be implemented before physical LVH occurs, to prevent loss of contractility through diminished voltage clamping in LVH, and subendocardial ischemia due to altered transmural perfusion, as well as arrhythmogenesis. Aggressive treatment of hypertension becomes an ever greater priority in this view.

Research on modes of injury to the His-Purkinje system other than strain from hypertension and LVH may show further ways to reduce the incidence of CHF and the attendant clinical burden and morbidity and mortality.

Drugs developed to act selectively at His-Purkinje cells to enhance conduction and depolarization might serve to enhance or preserve function in the Purkinje cell network, perhaps acting at the Purkinje gap junctions or sarcoplasmic reticulum, or by altering the level of expression of the calcium-or potassium-handling membrane proteins. This would potentially improve contractility in some weakened hearts without increasing ATP demand.

In established instances of LBBB, pacing into any surviving Purkinje cells distal to the block provides a way to increase contractility beyond the results provided by drugs. This would result from restoring laminar and lateral coordination. The LBBB caused by right ventricular pacing leads is functionally the same (30). Deshmukh et al (31) have shown that a transseptal pacing lead can be inserted from a venous approach and successfully activate the His bundle and left bundle; this restores a meaningful degree of contractility without generating additional metabolic demands, and without the complications of arterial or surgical approaches. This benefit has been demonstrated by Fei et al (32) with biventricular pacing leads in dogs, yielding a 10 per cent improvement in cardiac output over right ventricular pacing alone. Biventricular leads may be more useful if His pacing fails or if regional block occurs distal to the His bundle; transseptal insertion to the left ventricle would be desirable. Biventricular leads would also probably suppress arrhythmias in overdrive pacing more effectively, as the septum would be unavailable to circulate a purely left ventricular scroll wave; this technique might be superior to electrical cardioversion if leads were already in place.

Pacing into the right bundle could provide lateral and laminar coordination to the left ventricle if retrograde conduction in the right bundle can cross over to the left bundle. If the retrograde depolarization must pass through the AV node to access the left bundle, due to lack of collateral connections between the bundles, the node may block access to the left bundle. It appears that the right bundle is already excited in typical current pacemaker usage with right ventricular leads, since the resultant EKG pattern is typically LBBB, not trifascicular block. If this is the case, directly pacing into the left bundle itself is needed. By whatever means, successful activation of the full regulation of repolarization by all existent Purkinje fibers is needed to prevent pump deterioration and ultimately death.

References:

1. Wilson, FN, MacLeod AG, Barker PS: The T deflection of the electrocardiogram. Trans Assoc Am Physicians 1931;46:29.

2. Grant RP, Estes EH Jr: Spatial Vector Electrocardiography. Blakiston, 1951, p. 132-135.

3. Hellerstein, HK, Liebow IM: Factors influencing the T wave of the electrocardiogram. An experimental study employing intracavitary and extraventricular (epicardial) leads. Am Heart J 1950;39.35.

4. Adapted from Fruehan, CT, Crain BA, Burgess MJ, Millar K, Abildskov JA: Observations concerning the validity of the ventricular gradient concept. Am Heart J 1969;78(6):797, FIG. 1. Used by permission.

5. Wilson, FN, Finch R: The effect of drinking iced-water upon the form of the T deflection of the electrocardiogram. Heart 1923;10:275.

6. Pipeberger, H, Schwartz L, Massumi RA, Prinzmetal M: Studies on the nature of the repolarization process, XIX, Studies on the mechanism of ventricular activity. Am Heart J 1957;53(1):100.

7. Guyton AC: Textbook of Medical Physiology. W. B. Saunders, ed. 9, 1996, p. 124, paragraph 6.

8. Zipes DP: Genesis of cardiac arrhythmias: Electrophysiological considerations In Braunwald E, ed: Heart Disease. WB Saunders, ed. 5, 1997, ch. 20, p. 550, paragraph 6.

9. Garson, A: How to measure the QT interval—what is normal? Am J Cardiol 1993;72(6):14B.

10. Noble D, Bett G. Reconstructing the heart: a challenge for integrative physiology. Cardiovasc Res 1993;27:1701.

11. Noble D, Cohen I. The interpretation of the T wave of the electrocardiogram. Cardiovasc Res 1978;12:13.

12. Rumancik, MW, Denlinger JK, Nahrwold ML, Falk RB: The QT interval and serum ionized calcium. JAMA 1978;240(4):366.

13. Surawicz, B: Role of potassium channels in cycle length dependent regulation of action potential duration in mammalian cardiac Purkinje and ventricular muscle fibres. Cardiovasc Res 1992;26:1021.

14. Yokoshiki, H, Kohya T, Tomita F, Tohse N, Nakaya H,. Kanno M, Kitabatake A: Restoration of action potential duration and transient outward current by regression of left ventricular hypertrophy. J Mol Cell Cardiol 1997;29:1331.

15. Sperelakis N: Origin of the cardiac resting potential In Berne RM, ed: Handbook of Physiology. The Cardiovascular System. American Physiology Society; Bethesda MD, 1979, ch. 6, p. 190, Table 1.

16. Hoffman, BF, Kao CY, Suckling EE: Refractoriness in cardiac muscle. Am J Physiol 1957;190:473.

17. Viragh, S, Stoeckel ME, Porte A: Light and electron microscopic structure of the cardiac Purkinje fibers—Review. Physiologia Bohemoslovaca 1987;36:233.

18. Akhtar, M, Damato AN, Batsford WP, Ruskin JN, Ogunkelu JB: A comparative analysis of antegrade and retrograde conduction patterns in man. Circulation 1975;52:766.

19. El-Sherif, N, Chinushi M, Caref EB, Restivo M: Electrophysiological mechanism of the characteristic electrocardiographic morphology of torsades de pointes tachyarrhythmias in the long-QT syndrome. Circulation 1997;96(12):4392.

20. Zabel, M, Hohnloser SH, Behrens S, Li YG, Woosley RL, Franz MR: Electrophysiologic features of torsades de pointes. J Cardiovasc Electrophysiol 1997;8(10):1148.

21. Tzivoni, D, Keren A, Cohen AM, Loebel H, Zahavi I, Chenzbraun A, Stern S: Magnesium therapy for torsades de pointes. Am J Cardiol 1984;53:528.

22. Stark, G, Stark U, Pilger E, Honigl K, Bertruch H, Tritthart HA: The influence of elevated Mg++concentrations on cardiac electrophysiologic parameters. Cardiovasc Drugs Ther 1989;3:183.

23. Charpentier, F, Baudet S, Le Marec H: Triggered activity as a possible mechanism for arrhythmias in ventricular hypertrophy. Pacing Clin Electrophysiol 1991;14(11 Part 2):1735.

24. Hamlin, RL, Burton RR, Leverett SD, Burns JW: Ventricular activation process in minipigs. J Electrocardiol 1975;8(2):113.

25. Upshaw, CB: Seeing through the maze of complete left bundle branch block. J Med Assoc GA 1993;82:593.

26. Noma, A, Tsuboi N: Dependence ofjunctional conductance on proton, calcium and magnesium ions in cardiac paired cells of guinea-pig. J Physiol (Lond) 1987;382:193.

27. Bell, JR, Fox AC: Pathogenesis of subendocardial ischemia. Am J Med Sci 1974; 268(1):2.

28. Waller, BF, Gering LE, Branyas NA, Slack JD: Anatomy, histology, and pathology of the cardiac conduction system: Part II. Clin Cardiol 1993;16:347.

29. Fibrinolytic Therapy Trialists' Collaborative Group: Indications for fibrinolytic therapy in suspected acute myocardial infarction. Lancet 1994;343:311.

30. Tanabe, A, Mohri T, Ohga M, Yoshiga O, Hidaka Y, Ikeda H, Hiyamuta K, Koga Y, Toshima H: The effects of pacing-induced left bundle branch block on left ventricular systolic and diastolic performances. Jpn Heart J 1990;31(3):309.

31. Deshmukh, P, Casavant DA, Romanyshyn M, Anderson K: Permanent, direct His-bundle pacing, a novel approach to cardiac pacing in patients with normal His-Purkinje activation. Circulation 2000;101:869.

32. Fei, L, Wrobleski D, Groh W, Vetter A, Duffin EG, Zipes DP: Effects of multisite ventricular pacing on cardiac function in normal dogs and dogs with heart failure. J Cardiovasc Electrophysiol 1999;10(7):935.

TABLE I
Comparing characteristics of varying cell population
monophasic action potentials (MAP), and showing the
tracking of subendocardial MAP with Purkinje MAP.

| | DURATION, | |
|---|---|---|
| CELL TYPE | MSEC | COMMENTS |
| nerve | 0.3 | Most energy-efficient for |
| | | activation only. |
| Purkinje | 400 | Wastes energy if only |
| | | activation is accomplished. |
| subepicardial myocyte | 250 | Same with or without bundle |
| | | activation |
| isolated subendocardial | 250 | Identical to subepicardial; |
| myocyte | | native state. |
| Purkinje-activated | 400 | Identical to Purkinje; |
| subendocardial myocyte | | regulated state. |

[image]

What we claim as our invention is any and all means of enhancing cardiac function in the human by sustaining, preserving, or augmenting laminar coordination of ventricular myocardial repolarization by the His-Purkinje system:

1: We claim as our invention any application of electronic cardiac pacemaker or pacemakers for the purpose of sustaining, restoring, or enhancing laminar coordination of ventricular myocardial repolarization in all or part of either or both of the right or left cardiac ventricles by all or part of the His-Purkinje cell system.

2: We claim as our invention any application of any drug, chemical, or molecule, singly or in combination, for the purpose of sustaining, restoring, or enhancing laminar coordination of ventricular myocardial repolarization in all or part of either or both of the right or left cardiac ventricles by all or part of the His-Purkinje cell system, by any action on the sarcolemma, cytosol, sarcoplasmic reticulum, nucleus, or any other cellular organelles, in either the cells of the His-Purkinje system or the ventricular myocardial cells.
